(19) United States
(12) Patent Application Publication
(10) Pub. No.: US 2024/0199964 A1
(43) Pub. Date: Jun. 20, 2024

(54) COMPOUNDS FOR MARKING LIQUIDS

(71) Applicant: KUANTAG NANOTEKNOLOJILER GELISTIRME VE URETIM A.S., Istanbul (TR)

(72) Inventor: ZIYA KOSTERELI, ISTANBUL (TR)

(73) Assignee: KUANTAG NANOTEKNOLOJILER GELISTIRME VE URETIM A.S., ISTANBUL (TR)

(21) Appl. No.: 18/070,451

(22) Filed: Nov. 28, 2022

(51) Int. Cl. C10L 1/00; C09K 11/06; C10L 1/232; G01N 21/31; G01N 21/64; G01N 33/28

(52) U.S. Cl. CPC C10L 1/003; C09K 11/06; C10L 1/232; G01N 21/31; G01N 21/6428; G01N 33/28; C09K 2211/1007; C09K 2211/1014; C09K 2211/1022; C10L 2200/0446; C10L 2230/16; G01N 2021/6439

(57) ABSTRACT

Present invention provides a method for marking hydrocarbon fuels with a marker material and tracking the marked hydrocarbon fuel in a fuel distribution network. The marker material includes an anthrapyridone compound.

### FIELD

The present invention generally relates to fuel marking and tracking, more particularly, to use of anthrapyridone compounds and methods for marking and tracking of fuels.

### BACKGROUND

Today oil (gasoline or diesel fuel) is the fuel of choice for most of the transportation modes in the world. In fact, more than 50 percent of oil used around the world is consumed by the transportation sector. Approximately 75 percent of the oil consumed by overall transportation sector is in the field of road transportation. This is because oil is currently the only fuel having a distinctive combination of availability, portability, and affordability.

In many developed and developing countries, oil and gas industries are very important because excise tax revenues from fuel sales contribute their economies. Especially in growing economies, high excise tax can add up to the price of fuel.

Due to its monetary value and the transportation sector's dependence on fuel, fuel smuggling, fuel adulteration and fuel tax evasions have become a growing problem in some countries and pose serious threats to the revenues of such countries as well as energy companies worldwide.

The most common way of adulteration involves blending or diluting high quality branded fuel products with inferior products, such as diluting gasoline with cheaper kerosene. Estimated economic value of such improper actions is in the range of billions of USD per year. Therefore, fuel supply integrity and quality are of vital importance for fuel tax revenues.

Thus, it will become readily apparent that it would be highly desirable to provide marking and tracking methods to monitor fuel distribution networks effectively to protect the integrity and the quality of the distributed fuel.

### SUMMARY

The present inventions are related to fuel marking and tracking. An aspect of the present invention includes a marked hydrocarbon fuel including a hydrocarbon fuel; and a marker material including at least one derivative of anthrapyridone compound, wherein the anthrapyridone compound consists of the general formula (I)

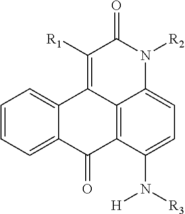

Wherein R1 represents hydrogen, or a substituted or unsubstituted alkyl, aryl, benzoyl, hetaryl, alkoxy or phenoxy group; R2 represents hydrogen, or a substituted or unsubstituted alkyl, alicyclic, aryl or, hetaryl group; R3 a substituted or unsubstituted alkyl, alicyclic, aryl, hetaryl, alkoxy, phenoxy, amino, or amido group.

Another aspect of the present invention includes a method of identifying a marked hydrocarbon fuel containing a marker material consisting of an anthrapyridone compound, the method includes analyzing a sample of the marked hydrocarbon fuel in a spectroscopic analyzer to identify the presence of the marker material in the marked hydrocarbon fuel; determining concentration of the marker material present in the sample of the marked hydrocarbon fuel; and wherein the anthrapyridone compound is selected from the group consisting of an anthrapyridone compound with formula C₂₃H₁₆N₂O₂, an anthrapyridone compound with formula C₂₃H₂₂N₂O₂, and combinations thereof.

Yet another aspect of the present invention provides a method of preparing and identifying a marked hydrocarbon fuel containing a marker material consisting of an anthrapyridone compound, the method includes preparing a marker solution comprising a solvent including the marker material in a predetermined concentration range; wherein the anthrapyridone compound is selected from the group consisting of an anthrapyridone compound with formula C₂₃H₁₆N₂O₂, an anthrapyridone compound with formula C₂₃H₂₂N₂O₂, and combinations thereof; mixing the marker solution with a hydrocarbon fueld to form the marked hydrocarbon fuel having an adjusted concentration of the marker material; analyzing a sample of the marked hydrocarbon fuel in a spectroscopic analyzer to identify the presence of the marker material; and determining the adjusted concentration of the marker material present in the sample of the marked hydrocarbon fuel.

### BRIEF DESCRIPTION OF THE DRAWINGS

FIG. 1 is a flowchart of an embodiment of a method of the present invention;

FIG. 2A is graph showing an exemplary absorbance spectrum of an anthrapyridone marker in diesel fuel; and

FIG. 2B is a graph showing an exemplary fluorescence emission spectrum of the anthrapyridone marker in diesel fuel.

### DETAILED DESCRIPTION

The present invention relates to materials for marking, tracking, and monitoring of liquids in order to identify them and /or to detect unwanted or illegal alterations during their transportation, storage or usage, and methods thereof. In some embodiments, exemplary liquids may be the following: petroleum products for example a hydrocarbon fuel such as gasoline, diesel fuel, or engine oil, or the like, and/or any combinations thereof; and organic solvents such as hydrocarbon-based, alcohol-based, ester-based, ether-based, ketone-based solvents.

In fuel industry, supply chain integrity, or tracking transported fuels, may be crucial to prevent fuel smuggling and fuel fraud. Many countries have national fuel marker programs and add fuel markers to commercial fuels to keep the fuel supply chain safe. In general, these markers may not be visible to the naked eye, difficult to detect and does not affect the fuel performance or properties. Some countries have national marker policies or programs, and it is legal to use markers in fuels in such countries. In addition to the national marker policies, private fuel suppliers can use their own fuel marker programs to track their fuel supply to keep the fuel quality at a standard level.

A fuel can be altered by being mixed, diluted, or adulterated with one or more other fuels, solvents, oils, petrochemicals, and any combination thereof. The term ‘fuel’ used herein is understood to mean any hydrocarbons, hydrocarbon fuels, hydrocarbon solutions, hydrocarbon fluids, petroleum based products, biofuels, fossil fuels, gasoline, diesel fuel, kerosene, engine oils, and the like. For the purposes of the present invention, the term “altered fuel” is understood to mean a fuel that has been illegally mixed, diluted, or adulterated by other liquids.

In one embodiment, the present invention provides a spectroscopically detectable marker material, or marker, to mark hydrocarbon fuels, i.e., mixing a preferred quantity of the marker material with a fuel of known quantity. An exemplary spectroscopically detectable marker material may be a fluorescent marker material, which may enable identification of the hydrocarbon fuel, when the marker material is qualitatively and quantitively detected. When a sample of the hydrocarbon fuel having the marker material, is exposed to a particular wavelength of light, i.e., spectroscopically analyzed in a spectroscope, the marker material may emit a signature radiation, or fluoresce, at a known wavelength enabling the marker material's spectroscopical identification in the hydrocarbon fuel sample.

In one embodiment of the present invention, an exemplary marker may be an organic compound comprised of anthrapyridone compound (crystal), or anthrapyridone dye. Anthrapyridone compounds have optical properties (UV and fluorescence) in visible region of light and are solvable in liquids, such as hydrocarbon-based, alcohol-based, ester-based, ether- based, ketone-based solvents, to be optically detected by a spectroscopic analysis. Because of their optical properties and solubility in liquids, anthrapyridone compounds of the present invention may advantageously be used as marker materials to mark liquids including hydrocarbon fuels.

Core chemical structure, or core chemical formula, of an anthrapyridone compound or anthrapyridone crystal is provided below and will be referred to as general formula (I) hereinafter.

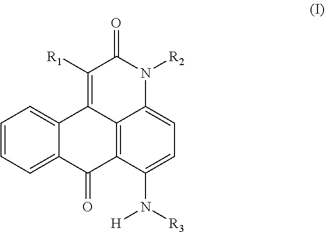

In the general formula (I) of anthrapyridone compound, R1 represents hydrogen, substituted alkyl group, substituted aryl group, substituted benzoyl group, substituted hetaryl group, substituted alkoxy group, substituted phenoxy group, unsubstituted alkyl group, unsubstituted aryl group, unsubstituted benzoyl group, unsubstituted hetaryl group, unsubstituted alkoxy group, or unsubstituted phenoxy group; R2 represents hydrogen, substituted alkyl group, substituted alicyclic group, substituted aryl group, substituted hetaryl group, unsubstituted alkyl group, unsubstituted alicyclic group, unsubstituted aryl group, or unsubstituted hetaryl group; and R3 represents substituted alkyl group, substituted alicyclic group, substituted aryl group, substituted hetaryl group, substituted alkoxy group, substituted phenoxy group, substituted amino group, substituted amido group, unsubstituted alkyl group, unsubstituted alicyclic group, unsubstituted aryl group, unsubstituted hetaryl group, unsubstituted alkoxy group, unsubstituted phenoxy group, unsubstituted amino group, or unsubstituted amido group.

An exemplary anthrapyridone marker compound (C₂₃H₁₆N₂O₂) shown below with formula (II) includes the anthrapyridone core chemical structure of the general formula (I), wherein R₁ is H, R₂ is CH₃, and R₃ is C₇H₇, which is commercially known as ‘Solvent Red 52’ and has a CAS number of ‘81-39-0’, the chemical structure of which is provided below and will be referred to as formula (II) hereinafter. The marker compound C₂₃H₁₆N₂O₂ has an absorption maximum of 543 nanometers (nm).

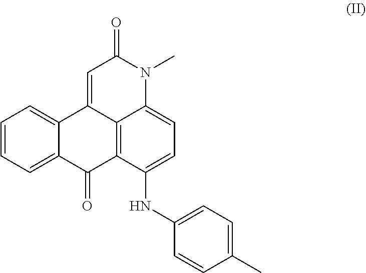 [SMILES: Cc1ccc(Nc2ccc3c4c(cc(=O)n3C)-c3ccccc3C(=O)c24)cc1]

Another exemplary anthrapyridone marker compound (C₂₃H₂₂N₂O₂) shown below with formula (III) includes the anthrapyridone core chemical structure of the general formula (I), wherein R₁ is H, R₂ is CH₃, and R₃ is C₆H₁₁, which is commercially known as ‘Solvent Red 149’and has a CAS number of ‘21295-57-8’, the chemical structure of which is provided below and will be referred to as formula (III) hereinafter. The marker compound C₂₃H₂₂N₂O₂ has an absorption maximum of 551 nm and an emission maximum of 580 nm.

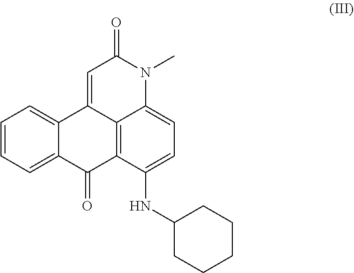 [SMILES: Cn1c(=O)cc2c3c(c(NC4CCCCC4)ccc31)C(=O)c1ccccc1-2]

Another exemplary anthrapyridone marker compound (C₂₄H₁₈N₂O₂) shown below with formula (IV) includes the anthrapyridone core chemical structure of the general formula (I), wherein R₁ is H, R₂ is CH₃, and R₃ is C₇H₇, which is commercially known as “Solvent Red 150’ and has a CAS number of 21295-58-9, the chemical structure of which is provided below and will be referred to as formula (IV) hereinafter. The marker compound C₂₄H₁₈N₂O₂ has an absorption maximum of 551 nm and an emission maximum of 580 nm.

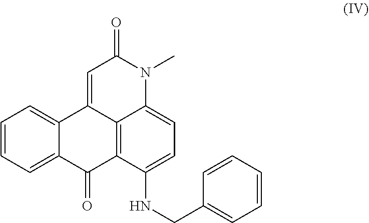 [SMILES: Cn1c(=O)cc2c3c(c(NCc4ccccc4)ccc31)C(=O)c1ccccc1-2]

Any anthrapyridone chemical structure meeting the criteria of the general formula (I) shown above may be used for marking the liquids, i.e., solvents, hydrocarbon fuels. In this respect, any derivative compound of the general formula (I) may be used as a marking material. The derivatives or compounds that are formed based on the general formula (I) has a common core chemical structure of anthrapyridone shown above. It may be possible to form or design a plurality of new compounds, or molecules, using the general formula (I). The general formula (I) shows the common core structure (anthrapyridone) and derivatization positions, commonly known as side groups (R₁, R₂ and R₃), to form new anthrapyridone based compounds, or derivatives of the common core structure (I).

Anthrapyridone-based marker compounds are compatible and stable in hydrocarbon fuels for marking purposes. For example, because of its optical properties, the anthrapyridone marker compound with formula (II) may not be affected from changing of the composition of the fuel (gasoline or diesel fuel) and/or additives added to the fuel. This feature provides robust and reproducible spectral readings resulting stable and reproducible quantification results.

Chemical structures of other examples of anthrapyridone marker compounds derived based on the general formula (I) may be seen below. Table 1 shows exemplary anthrapyridone compound markers (formulas (II)-(X)) that may be used as markers for marking hydrocarbon fuels. Table 1 identifies side groups of each anthrapyridone compound based on the general formula (I) and their absorption and emission characteristics. In Table 1, only the absorbance maximum and emission maximum values for the anthrapyridone compound markers with formula (II) and formula (III) are experimental. In the spectroscopic measurements, each test sample includes a xylene solution having 0.5 ppm marker material. For emission measurements 543 and 551 nm excitation wavelength are used, respectively. The absorbance maximum and emission maximum values of the formulas (IV)-(X) are not experimental values. These values are estimated values based on the values of the formulas (II) and (III).

TABLE 1

| | | | | Ab. max. | Em. max. |
|---|---|---|---|---|---|
| No. | R1-Position | R2-Position | R3-Position | (nm) | (nm) |
| (II) | H (Hydrogen) | CH₃ (methyl) | C₇H₇ (methyl benzene) | 543 | no emission |
| (III) | H (Hydrogen) | CH₃ (methyl) | C₆H₁₁ (cyclohexyl) | 551 | 580 |
| (IV) | H (Hydrogen) | CH₃ (methyl) | C₇H₇ (methyl benzene) | 540-560 | 575-600 |
| (V) | H (Hydrogen) | CH₃ (methyl) | CH₃ (methyl) | 540-560 | 575-600 |
| (VI) | H (Hydrogen) | C₈H₁₇ (2-ethyl-hexyl) | C₈H₁₇ (2-ethyl-hexyl) | 540-560 | 575-600 |
| (VII) | H (Hydrogen) | CH₃ (methyl) | C₈H₁₇ (2-ethyl-hexyl) | 540-560 | 575-600 |
| (VIII) | H (Hydrogen) | C₆H₁₁ (cyclohexyl) | C₆H₁₁ (cyclohexyl) | 540-560 | 575-600 |
| (IX) | H (Hydrogen) | CH₃ (methyl) | C₁₁H₂₃ (undecyl) | 540-560 | 575-600 |
| (X) | H (Hydrogen) | C₇H₇ (methyl benzene) | C₇H₇ (methyl benzene) | 540-560 | 575-600 |

Another exemplary anthrapyridone marker compound (C₁₈H₁₄N₂O₂) shown below with formula (V) includes the anthrapyridone core chemical structure of the general formula (I), wherein R₁ is H, R₂ is CH₃, and R₃ is CH₃, the chemical structure of which is provided below and will be referred to as formula (V) hereinafter. The marker compound C₁₈H₁₄N₂O₂ has an estimated absorption maximum range of 540-560 nm and an estimated emission maximum range of 575-600 nm.

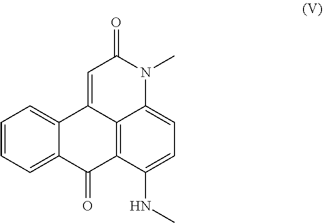 [SMILES: CNc1ccc2c3c(cc(=O)n2C)-c2ccccc2C(=O)c13]

Another exemplary anthrapyridone marker compound (C₃₂H₄₂N₂O₂) shown below with formula (VI) includes the anthrapyridone core chemical structure of the general formula (I), wherein R₁ is H, R₂ is C₈H₁₇, and R₃ is C₈H₁₇, the chemical structure of which is provided below and will be referred to as formula (VI) hereinafter. The marker compound C₃₂H₄₂N₂O₂ has an estimated absorption maximum range of 540-560 nm and an estimated emission maximum range of 575-600 nm.

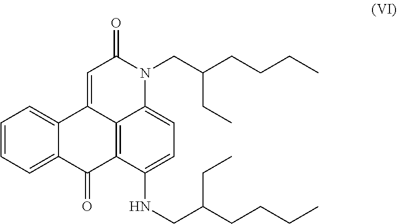 [SMILES: CCCCC(CC)CNc1ccc2c3c(cc(=O)n2CC(CC)CCCC)-c2ccccc2C(=O)c13]

Another exemplary anthrapyridone marker compound (C₂₅H₂₈N₂O₂) shown below with formula (VII) includes the anthrapyridone core chemical structure of the general formula (I), wherein R₁ is H, R₂ is CH₃, and R₃ is C₈H₁₇, the chemical structure of which is provided below and will be referred to as formula (VII) hereinafter. The marker compound C₂₅H₂₈N₂O₂ has an estimated absorption maximum range of 540-560 nm and an estimated emission maximum range of 575-600 nm.

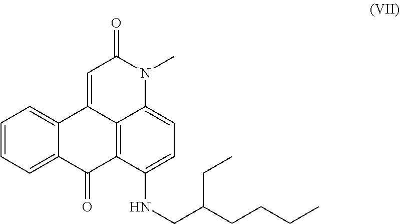 [SMILES: CCCCC(CC)CNc1ccc2c3c(cc(=O)n2C)-c2ccccc2C(=O)c13]

Another exemplary anthrapyridone marker compound (C₂₈H₃₀N₂O₂) shown below with formula (VIII) includes the anthrapyridone core chemical structure of the general formula (I), wherein R₁ is H, R₂ is C₆H₁₁, and R₃ is C₆H₁₁, the chemical structure of which is provided below and will be referred to as formula (VIII) hereinafter. The marker compound C₂₈H₃₀N₂O₂ has an estimated absorption maximum range of 540-560 nm and an estimated emission maximum range of 575-600 nm.

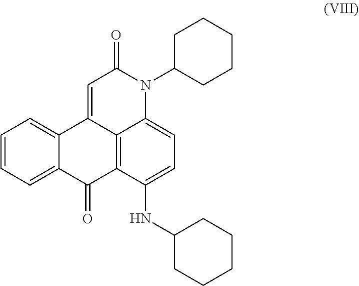 [SMILES: O=C1c2ccccc2-c2cc(=O)n(C3CCCCC3)c3ccc(NC4CCCCC4)c1c23]

Another exemplary anthrapyridone marker compound (C₂₈H₃₄N₂O₂) shown below with formula (IX) includes the anthrapyridone core chemical structure of the general formula (I), wherein R₁ is H, R₂ is CH₃, and R₃ is C₁₁H₂₃, the chemical structure of which is provided below and will be referred to as formula (IX) hereinafter. The marker compound C₂₈H₃₄N₂O₂ has an estimated absorption maximum range of 540-560 nm and an estimated emission maximum range of 575-600 nm.

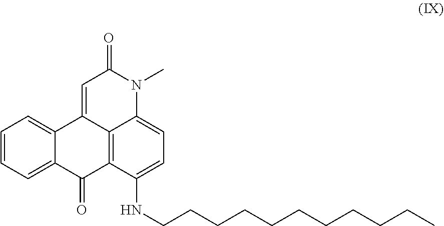 [SMILES: CCCCCCCCCCCNc1ccc2c3c(cc(=O)n2C)-c2ccccc2C(=O)c13]

Another exemplary anthrapyridone marker compound (C₃₀H₂₂N₂O₂) shown below with formula (X) includes the anthrapyridone core chemical structure of the general formula (I), wherein R₁ is H, R₂ is C₇H₇, and R₃ is C₇H₇, the chemical structure of which is provided below and will be referred to as formula (X) hereinafter. The marker compound C₃₀H₂N₂O₂ has an estimated absorption maximum range of 540-560 nm and an estimated emission maximum range of 575-600 nm.

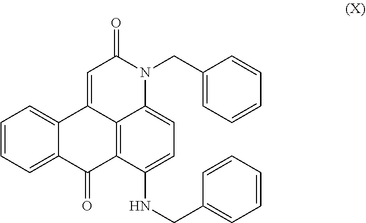 [SMILES: O=C1c2ccccc2-c2cc(=O)n(Cc3ccccc3)c3ccc(NCc4ccccc4)c1c23]

The flexibility of forming new compounds based on the core chemical structure of anthrapyridone (general formula (I)) may provide the following advantages: (1) being able to increase the stability and compatibility of the marker compound in the marked liquid (hydrocarbon fuels, or other liquids), i.e., making it more compatible to the environment where it will be used; and (2) being able to change the solubility of the marker compound in the marked liquid by changing the side groups (R₁, R₂ and R₃ groups of the general formula (I)). The solubility increase may provide economic advantages.

This way, using the general formula (I), various anthrapyridone compounds with desired marker material properties may be custom designed using this approach. Absorbance or fluorescence spectroscopy, or both, may be used to detect the anthrapyridone compounds disclosed in this disclosure. The only difference may be the absorbance or emission wavelengths detected with these spectroscopic processes.

For example, because the diesel fuel has more aliphatic nature compared to gasoline, it may be beneficial to design different anthrapyridone marker compounds for them. As described above, only the side groups (R₁, R₂ and R₃) may be changed to design the marker compound with desired properties including compatibility and solubility. A marker compound for marking the diesel fuel may include an aliphatic side groups (R₁, R₂ and R₃) and a marker compound for marking the gasoline may include aromatic side groups (R₁, R₂ and R₃). Such aliphatic and aromatic side groups may make the markers more compatible, such as optically, and more stable in diesel fuel and in gasoline, respectively.

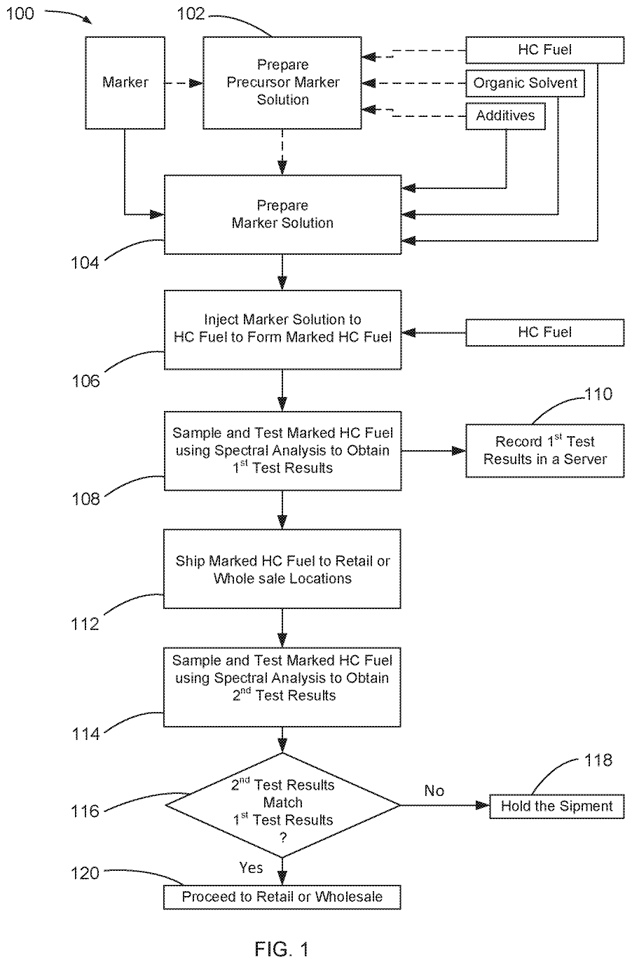

FIG. 1 is a flowchart 100 showing an embodiment of a method of marking a hydrocarbon fuel (HC fuel) with an anthrapyridone compound (marker material), such as the formula (III) described above, and tracking the distribution of the marked HC fuel, for example, until a retail place such as a gas station or a retail storage location, or a desired location.

In step 102, a precursor marker solution (PMS) may be prepared to be used in the preparation of the marker solution (next step 104). In one method, the precursor marker solution may be prepared by dissolving the marker material (high purity powder) either in the HC fuel to be marked, or in a liquid solvent, preferably a liquid organic solvent in step 102. In another method, the precursor marker solution may be prepared by dissolving the marker material (high purity marker material powder) in a solution containing both the HC fuel and a liquid organic solvent to form the precursor marker solution. In another method, the marker material, prepared in powder form (high purity), may be added to a liquid organic solvent such as xylene or benzyl alcohol at very high concentrations, i.e., greater than 20%, in step 102, to form a precursor marker solution. Next, this precursor marker solution, which is an organic liquid solution having the high concentration marker material is diluted in HC fuel to prepare a marker solution in the next step. Additives may also be added to the precursor marker solution prepared by any of these methods in step 102.

Exemplary organic solvents may be aromatic solvents including toluene, xylene, ethyl benzene; alcohol-based solvents including methyl alcohol, ethyl alcohol, isopropyl alcohol, and benzyl alcohol; and other organic solvents including ethyl acetate, acetone, methyl ethyl ketone, hexane, and cyclohexane. Organic solvents used for preparing precursor marker solution and marker solution may preferably be selected from the organic solvents that may not optically interfere with the optical response of the anthrapyridone compound marker. For example, optical response of the anthrapyridone compound marker in pure xylene solvent may be read without any optical interference (background interference) from xylene solvent, because xylene solvent has no absorbance or emission response in the optical response region of the anthrapyridone marker material. As a result, the detected optical response of the marker in xylene is substantially from the marker material. When organic solvents, or the fuel to be marked, are used as a solvent to prepare a precursor marker solution or a marker solution, both solutions may have 0.1-10 ppm, or 1-50 ppm marker material concentration.

In step 104, in a first method, a marker solution (MS) may be prepared by directly adding the marker material to the HC fuel to form the marker solution. Next, if needed, diluting the marker solution with a predetermined amount of HC fuel to obtain a desired marker concentration level. The marker solution may include an organic solvent and additives. In a second method, in step 104, the precursor marker solution prepared in step 102 may be mixed with predetermined amount of HC fuel to prepare a marker solution with a desired marker material concentration level. In this embodiment, other organic solvents and additives may be added to the marker solution during preparation or come with the precursor marker solution prepared in step 102. In both marker solution preparation methods, the marker solution should be stable at least 6 months in storage, i.e., it should not precipitate or lose its optical properties while in storage. In an alternative method, in step 104, the marker solution may be prepared by adding the marker material to a solvent, which may or may not include additives.

Once it is prepared in step 104, in step 106, the marker solution may be injected (added or mixed) into the HC fuel to prepare marked HC fuel. The marked HC fuel may have a marker material concentration at low ppm levels, such as about 0.1 ppm, or 0.2 ppm, or about 0.3 ppm, or about 0.5 ppm, or 0.6 ppm, or in the range of about 0.1-10 ppm, or about 0.1-0.6 ppm, or about 0.1-0.5 ppm, or about 0.2-0.5 ppm, or about 0.2-0.3 ppm.

In step 106, the injection of the marker solution into the HC fuel may be made automatically using an injection system or manually, i.e., by pouring a predetermined amount of the marker solution into a tank containing the HC fuel to be marked. Additives to be added to the marker HC fuel, may be either added to the precursor solution in step 104 or the precursor marker solution in step 102 to inject both the fuel marker material and fuel additives at the same time.

In step 108, the marked HC fuel may be sampled and tested to detect the marker material, in the marked HC fuel, qualitatively and quantitatively. As will be described more fully below, the test may be a spectroscopic analysis using UV-Vis or fluorescence spectroscopy. The resulting test data, such as 1st test results, may be recorded on a data storage of a computer system including a processor, in step 110. In step 108, concentration of the marker material present in the HC fuel is also determined and recorded. The marker material (anthrapyridone compound) and its concentration in the HC fuel is the identification, or ID tag, of the marked HC fuel to track.

Portable or lab scale optical spectroscopies may be used for the detection of the marker material and determination of the marker material concentration. The identity and quantity of the marker material may also be detected by advanced analytical techniques such as HPLC, GC using the optical properties of the anthrapyridone marker compounds used in the process.

In step 112, the marked HC fuel is transported to fuel tanks of a sale location which may be a retail or wholesale location, for example a gas station.

In step 114, the marked HC fuel may be again sampled and tested at the sale location by using the UV-Vis or fluorescence spectroscopy, and a test result data, such as 2^{nd} test results including observed marker material concentration, may be stored on a data storage of the same computer system.

In step 116, if both test results match, the marked HC fuel may proceed for sale. If the results don't match the shipped HC fuel is held for further inspection, e.g., differing marker material concentration. Additionally, if the recorded values are very close to each other, for example within 5% deviation or less than 5% deviation, the tested HC fuel may pass the test and may proceed for sale.

The flexibility of the above mentioned custom designing of the marker compounds may also provide economic advantages. For example, because of the marker compound's solubility limit, a marker solution supplied for marking a diesel fuel shipment may include 1% anthrapyridone marker compound for marking and tracking the shipped diesel fuel. As described above, changing the side groups (R₁, R₂ and R₃) with more soluble chemicals, i.e., longer aliphatic chains, the solubility of the marker compound may be increased at least 5%. This feature of the anthrapyridone compounds (marker material) of the present invention may reduce the amount of solvents and additives used in the marker solution preparation, resulting in reductions in costs of fuel shipment or transportation.

Similar processes may be applied to mark other liquids by changing some factors depending on the solvent or the liquid to be marked, and marker concentration.

The examples of organic solvent(s) that may be used as solvents to prepare marker solution (main stock marker solution) and precursor marker solution may be aromatic solvents, alcohol-based solvents, aliphatic solvents, fuels, and refinery products. More specifically, organic solvents may include aromatic solvents such as aromatic solvents such as toluene, xylene, ethyl benzene; alcohol-based solvents such as methyl alcohol, ethyl alcohol, isopropyl alcohol, butyl alcohol, benzyl alcohol, ethylene glycol, glycerin; ether-based solvents such as tetrahydrofuran, dioxane, ethylene glycol monomethyl ether; ester-based solvents such as ethyl acetate; ketone-based solvents such as cyclohexanone, acetone, methyl ethyl ketone; hydrocarbon-based solvents such as hexane, cyclohexane, toluene, xylene, mineral spirit, oil-based products such as gasoline, diesel, kerosene, naphtha, reformate.

To improve the stability and performance of the marker solution, additives, or chemicals, may also be used. Exemplary additives may be oleic acid and/or ethylene glycol. The anthrapyridone marker content of the marker solution may be in the range of about 0.1-50% (marker weight/solvent weight).

The anthrapyridone markers with the formulas II-X described above may be used with other materials, i.e., chemical, dyes, markers, as hybrid markers. Other materials may be directly added to the marker solution or the precursor marker solution. Alternatively, they may be prepared in a separate container as another marker solution before marking the fuel to be tracked. The materials may be other optical dyes and optical materials like organic and/or inorganic quantum dots. Optical dyes with commercial names Solvent Blue 35 (CAS No: 17354-14-2), Solvent Blue 97 (CAS No: 32724-62-2) and Solvent Red 164 (CAS No. 71819-51-7) may be given as other examples to be coupled with the anthrapyridone markers. Exemplary quantum dots may be carbon quantum dots and CdSe quantum dots.

In another hybrid marker approach, anthrapyridone markers of the present invention, which are optical markers, may also be used together with other chemicals not having optical properties. Such chemicals with no optical properties may be molecular markers such as benzyl alcohol. Due to their optical properties, anthrapyridone markers are easy to detect via spectroscopic techniques compared to the molecular markers. Molecular markers (so called silent or forensic markers) may generally be analyzed using chromatographic techniques such as Gas Chromatographic Mass Spectroscopy (GC-MS). If the anthrapyridone markers and molecular markers are used together to mark a fuel to be tracked, both analysis techniques (spectroscopic and chromatographic) may be used. For example, benzyl alcohol may be detected and tracked as a molecular marker in the marked fuel along with an anthrapyridone marker. In an alternative hybrid marking approach, more than one anthrapyridone compound type may be added to the fuel with or without molecular markers.

Accordingly, in the marked hydrocarbon fuel, the marker material may consist of one anthrapyridone compound, or more than one anthrapyridone compounds. Alternatively, in alternative embodiments, in the marked hydrocarbon fuel, the marker material may consist of a hybrid marker material including one or more anthrapyridone compounds and other marker materials that described above.

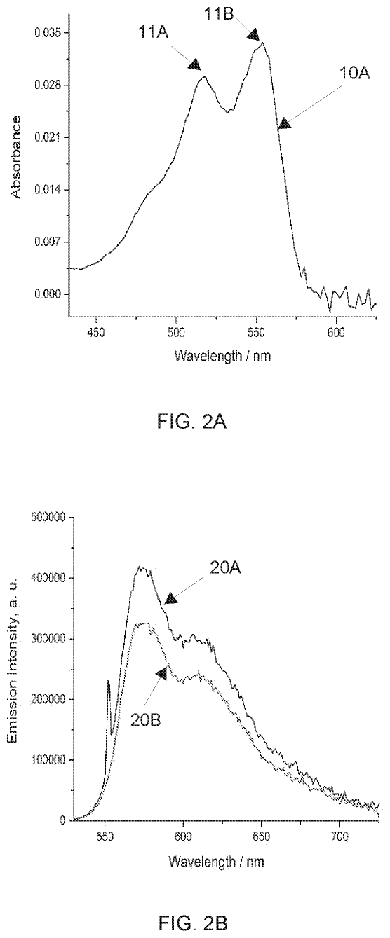

Absorbance or fluorescence spectroscopy, or both spectroscopies, may be used to detect and quantify the marker material in a marked hydrocarbon fuel to identify it. The optical properties of the anthrapyridone compound with the formula (III) in diesel fuel may be exemplified in FIGS. 2A-2B. The marked diesel fuel may be the exemplary marked HC fuel as described above in connection with FIG. 1.

FIG. 2A is a graph of an absorbance spectrum 10A of the marker material (the anthrapyridone compound of formula (III)) in diesel fuel. The absorbance spectrum 10A includes two absorbance maximums 11A and 11B at about 520 nm and about 550 nm wavelength values of the visible radiation range, respectively. A diesel solution having about 0.5 ppm marker material concentration may be used as a test sample for this spectral analysis. The absorbance spectroscopy instrument may be any type of absorbance spectroscopy instrument that is able to take absorbance spectrum in visible radiation region, i.e., 400-600 nm.

During an exemplary tracking operation, absorbance spectroscopy analysis may be used to test samples taken from the marked diesel fuel for quantification of the marker material. The absorbance value at about 550 nm (absorbance maximum) may be used to quantify the amount of marker material using a pre-determined model obtained from a calibration curve. The calibration curve may be prepared using marked diesel fuel samples with varying marker material concentrations.

In order to prepare the model, first, marked diesel fuel samples including different amount of marker material may be prepared. If the marker material concentration in a target diesel fuel sample is 0.5 ppm, calibration curve study samples may include six marked diesel samples including 0.4 ppm, 0.45 ppm, 0.5 ppm, 0.55 ppm, 0.6 ppm, and 0.65 ppm marker material concentrations. Each calibration curve sample is measured under the same absorbance spectroscopy conditions as the target sample with 0.5 ppm concentration (the marker material concentration used to identify the marked diesel fuel). During the following analysis, the absorbance value (absorbance maximum) at 550 nm is recorded for each calibration sample. Next, a calibration curve is formed based on maximum absorbance values versus marker concentration, and from the calibration curve, a linear model is obtained. The linear model is used to quantify the amount of marker in each tested diesel fuel sample to detect deviations from the target marker concentration (tracked marker concentration) in the marked diesel fuel to detect any suspicious activity, or to test an unknown marked diesel fuel. The same approach may be used for gasoline or other fuels.

FIG. 2B is a graph showing fluorescence spectrums 20A and 20B of the marker material, which is the anthrapyridone compound of formula (III) in diesel fuel. The fluorescence spectrum 20A has an excitation wavelength (λ_{exc}) of about 550 nm, and the fluorescence spectrum 20B has an excitation wavelength (λ_{exc}) of about 520 nm. A diesel fuel solution having about 0.5 ppm marker material concentration is also used as a test sample for this spectral analysis.

Fluorescence spectroscopy may also be used to quantify the amount of marker material in the marked diesel fuel sample. During the fluorescence spectroscopy excitation radiation wavelength causing the emission may be in a range of about 450-590 nm, and the collected emission radiation wavelength may be in the range of about 530-700 nm. Alternatively, radiation (light) wavelengths corresponding to the absorbance maximum values may be used to excite the sample, which may be about 520 nm and about 550 nm. Although the emission maximum value is the preferred emission value, any excitation radiation wavelength in the range of 450-590 nm and emission wavelength value in the range of 550-680 nm may be used. Preferably, 550 nm excitation wavelength and 600 nm emission wavelength may be used. As explained above for the absorbance calibration curve for a marked diesel fuel, by establishing the same with emission values versus marker material concentrations, an emission calibration curve which is a quantification model of the marker material, to detect deviations from the target marker concentration in the marked diesel fuel or to determine marker material concentration in an unknown marked fuel, may be established.

The absorbance spectrum shown in FIG. 2A is taken by a UV-Vis Spectroscopy instrument from Thermo Scientific Co in a test range of about 400-650 nm. The fluorescence spectrum shown in FIG. 2B is taken by a fluorescence spectroscopy instrument from Edinburgh Co.

The active anthrapyridone marker concentration in the diesel fuel may be in the range of about 0.3-0.5 ppm. The final concentration of the active marker compound in the fuel is decided after laboratory stability and compatibility tests according to the working fuel type and environment.

### EXAMPLE 1

A 20,000 ppm (2%) precursor marker solution (PMS) (FIG. 1) was prepared by dissolving 2 gram (g) of marker material (anthrapyridone compound of formula (III)) in 98 g of xylene (100% xylene). Next, a marker solution (MS) (FIG. 1) was prepared by mixing 0.2 g of PMS and 99.8 g diesel fuel. In this state, MS contained 2,000 ppm of marker material. 250 ppm MS was then injected into diesel fuel to complete marking of diesel fuel. After the injection, the diesel fuel (marked fuel) contained 0.5 ppm marker material concentration. The marked fuel was then sampled and analyzed using fluorescence spectroscopy (excitation wavelength, λ_{exc}: 518 nm and emission wavelength, λ_{exc}: 600 nm). The emission value at 600 nm is read and added to the pre-studied model to quantify the marker concentration in the sample.

### EXAMPLE 2

A 20,000 ppm (2%) precursor marker solution (PMS) (FIG. 1) was prepared by dissolving 2 g of marker material (anthrapyridone compound of formula (III)) in 73.5 g of xylene and 24.5 g of benzyl alcohol (75:25 ratio of xylene/benzyl alcohol). Next, a marker solution (MS) (FIG. 1) was prepared by mixing 0.2 g of PMS and 99.8 g diesel fuel. In this state, MS contained 2,000 ppm of marker material. Next, 250 ppm MS was injected into diesel fuel to complete marking of diesel fuel. After the injection, the diesel fuel (marked fuel) contained 0.5 ppm marker material concentration. The marked diesel fuel was then sampled and analyzed using fluorescence spectroscopy (with excitation wavelength, λ_{exc}: 518 nm and emission wavelength, λ_{exc}: 600 nm). The emission value at 600 nm is read and added to the pre-studied model to quantify the marker concentration in the sample.

### EXAMPLE 3

A 20,000 ppm (2%) precursor marker solution (PMS) (FIG. 1) was prepared by dissolving 2 g of marker material (anthrapyridone compound of formula (III)) in 49 g of xylene and 49 g of benzyl alcohol (50:50 ratio of xylene/benzyl alcohol). Next, a marker solution (MS) (FIG. 1) was prepared by mixing 0.2 g of PMS and 99.8 g diesel fuel. In this state, MS contained 2,000 ppm of marker material. Next, 250 ppm MS was injected into diesel fuel to complete marking of diesel fuel . After the injection, the diesel fuel (marked fuel) contained 0.5 ppm marker material concentration. The marked diesel fuel was then sampled and analyzed using fluorescence spectroscopy (with excitation wavelength, λ_{exc}: 518 nm and emission wavelength, λ_{exc}: 600 nm). The emission value at 600 nm is read and added to the pre-studied model to quantify the marker concentration in the sample.

### EXAMPLE 4

A 20,000 ppm (2%) precursor marker solution (PMS) (FIG. 1) was prepared by dissolving 2 g of marker material (anthrapyridone compound of formula (III)) in 24.5 g of xylene and 73.5 g of benzyl alcohol (25:75 ratio of xylene/benzyl alcohol). Next, a marker solution (MS) (FIG. 1) was prepared by mixing 0.2 g of PMS and 99.8 g diesel fuel. In this state, MS contained 2,000 ppm of marker material. Next, 250 ppm MS was injected into diesel fuel to complete marking of diesel fuel. After the injection, the diesel fuel (marked fuel) contained 0.5 ppm marker material concentration. The marked fuel was then sampled and analyzed using fluorescence spectroscopy (with excitation wavelength, λ_{exc}: 518 nm and emission wavelength, λ_{exc}: 600 nm). The emission value at 600 nm is read and added to the pre-studied model to quantify the maker concentration in the sample.

### EXAMPLE 5

A 20,000 ppm (2%) precursor marker solution (PMS) (FIG. 1) was prepared by dissolving 2 g of marker material (anthrapyridone compound of formula (III)) in 98 g of benzyl alcohol (100% benzyl alcohol). Next, a marker solution (MS) (FIG. 1) was prepared by mixing 0.2 g of PMS and 99.8 g diesel fuel. In this state, MS contained 2,000 ppm of marker material. Next, 250 ppm MS was injected into diesel fuel to complete marking of diesel fuel. After the injection, the diesel fuel (marked fuel) contained 0.5 ppm marker material concentration. The marked fuel was then sampled and analyzed using fluorescence spectroscopy (with excitation wavelength, λ_{exc}: 518 nm and emission wavelength, λ_{exc}: 600 nm). The emission value at 600 nm is read and added to the pre-studied model to quantify the marker concentration in the sample.

### EXAMPLE 6

A 20,000 ppm (2%) precursor marker solution (PMS) (FIG. 1) was prepared by dissolving 2 g of marker material (anthrapyridone compound of formula (III)) in 98 g of xylene (100% xylene). Next, a marker solution (MS) (FIG. 1) was prepared by mixing 0.2 g of PMS and 99.8 g diesel fuel. In this state, MS contained 2,000 ppm of marker material. Next, 200 ppm MS was injected into diesel fuel to complete marking of diesel fuel. After the injection, the diesel fuel (marked fuel) contained 0.4 ppm marker material concentration. The marked fuel was then sampled and analyzed using fluorescence spectroscopy (with excitation wavelength, λ_{exc}: 518 nm and emission wavelength, λ_{exc}: 600 nm). The emission value at 600 nm is read and added to the pre-studied model to quantify the marker concentration in the sample.

### EXAMPLE 7

A 20,000 ppm (2%) precursor marker solution (PMS) (FIG. 1) was prepared by dissolving 2 g of marker material (anthrapyridone compound of formula (III)) in 73.5 gram of xylene and 24.5 gram of benzyl alcohol (75:25 ratio of xylene/benzyl alcohol). Next, a marker solution (MS) (FIG. 1) was prepared by mixing 0.2 g of PMS and 99.8 g diesel fuel. In this state, MS contained 2,000 ppm of marker material. Next, 200 ppm MS was injected into diesel fuel to complete marking of diesel fuel. After the injection, the diesel fuel (marked fuel) contained 0.4 ppm marker material concentration. The marked fuel was then sampled and analyzed using fluorescence spectroscopy (with excitation wavelength, λ_{exc}: 518 nm and emission wavelength, λ_{exc}: 600 nm). The emission value at 600 nm is read and added to the pre-studied model to quantify the marker concentration in the sample.

### EXAMPLE 8

A 20,000 ppm (2%) precursor marker solution (PMS) (FIG. 1) was prepared by dissolving 2 g of marker material (anthrapyridone compound of formula (III)) in 98 g of xylene (100% xylene). Next, a marker solution (MS) (FIG. 1) was prepared by mixing 0.2 g of PMS and 99.8 g diesel fuel. In this state, MS contained 2,000 ppm of marker material. Next, 250 ppm MS was injected into diesel fuel to complete marking of diesel fuel. After the injection, the diesel fuel (marked fuel) contained 0.5 ppm marker material concentration. The marked fuel was then sampled and analyzed using fluorescence spectroscopy (with excitation wavelength, λ_{exc}: 518 nm, emission wavelength, λ_{exc}: 600 nm). The emission value at 600 nm is read and added to the pre-studied model to quantify the marker concentration in the sample

### EXAMPLE 9

A 20,000 ppm (2%) precursor marker solution (PMS) (FIG. 1) was prepared by dissolving 2 g of marker material (anthrapyridone compound of formula (III)) in 73.5 gram of xylene and 24.5 gram of benzyl alcohol (75:25 ratio of xylene/benzyl alcohol). Next, a marker solution (MS) (FIG. 1) was prepared by mixing 0.2 g of PMS and 99.8 g diesel fuel. In this state, MS contained 2,000 ppm of marker material. Next, 250 ppm MS was injected into diesel fuel to complete marking of diesel fuel. After the injection, the diesel fuel (marked fuel) contained 0.5 ppm marker material concentration. The marked fuel was then sampled and analyzed using fluorescence spectroscopy (with excitation wavelength, λ_{exc}: 518 nm and emission wavelength, λ_{exc}: 600 nm). The emission value at 600 nm is read and added to the pre-studied model to quantify the marker concentration in the sample.

### EXAMPLE 10

A 20,000 ppm (2%) precursor marker solution (PMS) (FIG. 1) was prepared by dissolving 2 g of marker material (anthrapyridone compound of formula (III)) in 49 g of xylene and 49 g of benzyl alcohol (50:50 ratio of xylene/benzyl alcohol). Next, a marker solution (MS) (FIG. 1) was prepared by mixing 0.2 g of PMS and 99.8 g diesel fuel. In this state, MS contained 2,000 ppm of marker material. Next, 250 ppm MS was injected into diesel fuel to complete marking of diesel fuel. After the injection, the diesel fuel (marked fuel) contained 0.5 ppm marker material concentration. The marked fuel was then sampled and analyzed using fluorescence spectroscopy (with excitation wavelength, λ_{exc}: 518 nm and emission wavelength, λ_{exc}: 600 nm). The emission value at 600 nm is read and added to the pre-studied model to quantify the marker concentration in the sample.

Although aspects and advantages of the present invention are described herein with respect to certain preferred embodiments, modifications of the preferred embodiments will be apparent to those skilled in the art. Thus, the scope of the present invention should not be limited to the foregoing discussion but should be defined by the appended claims.

I claim:

1. A marked hydrocarbon fuel, comprising:
a hydrocarbon fuel; and
a marker material consisting of at least one derivative of anthrapyridone compound, wherein the anthrapyridone compound consists of the general formula (I) 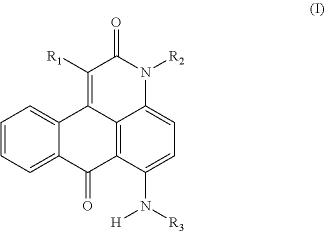
wherein R1 is hydrogen, substituted alkyl group, substituted aryl group, substituted benzoyl group, substituted hetaryl group, substituted alkoxy group, substituted phenoxy group, unsubstituted alkyl group, unsubstituted aryl group, unsubstituted benzoyl group, unsubstituted hetaryl group, unsubstituted alkoxy group, or unsubstituted phenoxy group; R2 is hydrogen, substituted alkyl group, substituted alicyclic group, substituted aryl group, substituted hetaryl group, unsubstituted alkyl group, unsubstituted alicyclic group, unsubstituted aryl group, or unsubstituted hetaryl group; and R3 is substituted alkyl group, substituted alicyclic group, substituted aryl group, substituted hetaryl group, substituted alkoxy group, substituted phenoxy group, substituted amino group, substituted amido group, unsubstituted alkyl group, unsubstituted alicyclic group, unsubstituted aryl group, unsubstituted hetaryl group, unsubstituted alkoxy group, unsubstituted phenoxy group, unsubstituted amino group, or unsubstituted amido group.

2. The marked hydrocarbon fuel of claim 1, wherein the at least one derivative of anthrapyridone compound consists of formula (II)

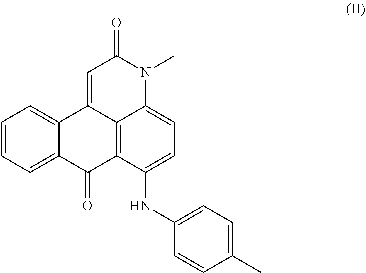 [SMILES: Cc1ccc(Nc2ccc3c4c(cc(=O)n3C)-c3ccccc3C(=O)c24)cc1]

3. The marked hydrocarbon fuel of claim 1, wherein the at least one derivative of anthrapyridone compound consists of formula (III)

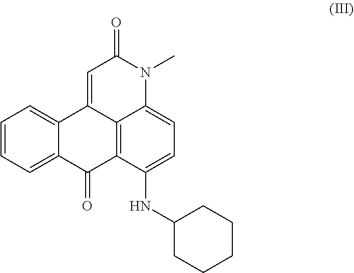 [SMILES: Cn1c(=O)cc2c3c(c(NC4CCCCC4)ccc31)C(=O)c1ccccc1-2]

4. The marked hydrocarbon fuel of claim 1, wherein the hydrocarbon fuel comprises one of gasoline, diesel fuel, and kerosene.

5. The marked hydrocarbon fuel of claim 1 further comprising at least one additive, wherein the at least one additive includes at least one of oleic acid and ethylene glycol.

6. The marked hydrocarbon fuel of claim 1, wherein a concentration of the marker material is in the range of 0.2-0.5 ppm.

7. A method of identifying a marked hydrocarbon fuel containing a marker material consisting of an anthrapyridone compound, the method comprising:
analyzing a sample of the marked hydrocarbon fuel in a spectroscopic analyzer to identify the presence of the marker material in the marked hydrocarbon fuel; and
determining concentration of the marker material present in the sample of the marked hydrocarbon fuel;
wherein the anthrapyridone compound is selected from the group consisting of an anthrapyridone compound of formula (II) 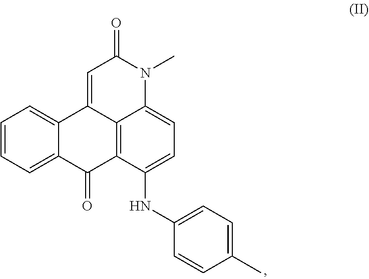 [SMILES: Cc1ccc(Nc2ccc3c4c(cc(=O)n3C)-c3ccccc3C(=O)c24)cc1]
an anthrapyridone compound of formula (III) 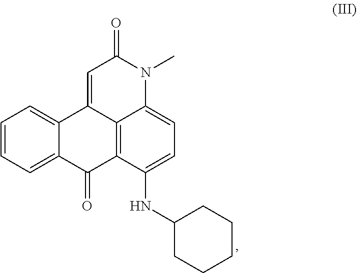 [SMILES: Cn1c(=O)cc2c3c(c(NC4CCCCC4)ccc31)C(=O)c1ccccc1-2]
and combinations thereof.

8. The method of claim 7, wherein the anthrapyridone compound is the anthrapyridone compound of formula (II).

9. The method of claim 7, wherein the anthrapyridone compound is the anthrapyridone compound of formula (III).

10. The method of claim 7, wherein the marked hydrocarbon fuel comprises one of gasoline, diesel fuel, and kerosene.

11. The method of claim 7, wherein the concentration of the marker material is in the range of 0.2-0.5 ppm.

12. The method of claim 7, wherein the spectroscopic analyzer is at least one of absorption spectroscope and emission spectroscope.

13. A method of preparing and identifying a marked hydrocarbon fuel containing a marker material consisting of an anthrapyridone compound, the method comprising:
preparing a marker solution comprising a solvent including the marker material in a predetermined concentration range;
wherein the anthrapyridone compound is selected from the group consisting of an anthrapyridone compound of formula (II) 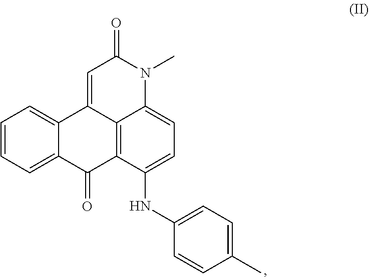 [SMILES: Cc1ccc(Nc2ccc3c4c(cc(=O)n3C)-c3ccccc3C(=O)c24)cc1]
an anthrapyridone compound of formula (III) 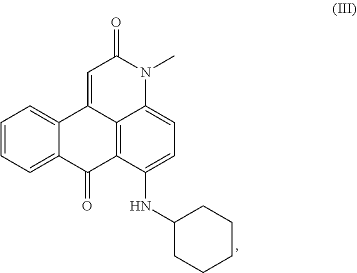 [SMILES: Cn1c(=O)cc2c3c(c(NC4CCCCC4)ccc31)C(=O)c1ccccc1-2]
and combinations thereof;
mixing the marker solution with a hydrocarbon fueld to form the marked hydrocarbon fuel having an adjusted concentration of the marker material;
analyzing a sample of the marked hydrocarbon fuel in a spectroscopic analyzer to identify the presence of the marker material; and
determining the adjusted concentration of the marker material present in the sample of the marked hydrocarbon fuel.

14. The method of claim 13, wherein the solvent is at least one of the hydrocarbon fuel and an organic solvent, wherein the organic solvent includes at least one of an aromatic solvent, an alcohol-based solvent and other organic solvent.

15. The method of claim 13, wherein the hydrocarbon fuel is one of gasoline, diesel fuel and kerosene.

16. The method of claim 13, wherein the adjusted concentration of the marker material is in the range of about 0.2-0.5 ppm.

17. The method of claim 14, wherein the aromatic solvent includes toluene, xylene, or ethyl benzene.

18. The method of claim 14, wherein the alcohol-based solvent includes methyl alcohol, ethyl alcohol, isopropyl alcohol, or benzyl alcohol.

19. The method of claim 14, wherein the other organic solvent includes ethyl acetate, acetone, methyl ethyl ketone, hexane, or cyclohexane.

20. The method of claim 13, wherein the marker solution further comprises an additive including at least one of oleic acid and ethylene glycol.

21. The method of claim 13, wherein the spectroscopic analyzer is at least one of absorption spectroscope and emission spectroscope.
